(19) United States
(12) Patent Application Publication
(10) Pub. No.: US 2024/0221877 A1
(43) Pub. Date: Jul. 4, 2024

(54) SYSTEMS AND METHODS FOR RANKING CLINICAL CONTEXT DOCUMENTS

(71) Applicant: GE Precision Healthcare LLC, Wauwatosa, WI (US)

(72) Inventors: Lino Martin Ramirez Velasquez, Concord, CA (US); John Vaughan Dwyer, Oak Park, IL (US); Praveen Babu Kysetti, Naperville, IL (US); Michael J. Mercado, Bartlett, IL (US)

(21) Appl. No.: 18/092,018

(22) Filed: Dec. 30, 2022

(51) Int. Cl. G16H 10/60

(52) U.S. Cl. CPC G16H 10/60

(57) ABSTRACT

A system may include a processor-based device configured to store or access a ranking application. When executed on or in communication with the workstation, the ranking application may determine a purpose of an exam request based on one or more attributes of the exam, retrieve a plurality of documents from a database based on the determined purpose, and assign a relevancy score to each of the plurality of documents based on a user profile. The ranking application may also create a list comprising the plurality of documents, wherein an order of the plurality of documents is based on the relevancy score and populate a graphical user interface (GUI) with the list for display on the workstation.

### BACKGROUND

The subject matter disclosed herein relates to medical data, and more particularly, to systems and methods for generating a personalized or otherwise customized list of clinical context documents to streamline a review process for making a diagnosis.

Non-invasive imaging technologies allow images of the internal structures or features of a subject (e.g., a patient, manufactured good, baggage, package, or passenger) to be obtained non-invasively. In particular, such non-invasive imaging technologies rely on various physical principles, such as the differential transmission of X-rays through the target volume or the reflection of acoustic waves, to acquire data and to construct images or otherwise represent the internal features of the subject. By way of example, in X-ray based imaging technologies, signals representative of an amount or an intensity of radiation may be collected and the signals may then be processed to generate an image that may be processed or displayed for review.

When reviewing medical images, radiologists may first review clinical information (e.g., patient history, case information) to get an understanding of the reason for an exam. In certain instances, the information provided by a referring physician may be vague regarding the reason (e.g., purpose) for ordering the exam. As such, the radiologist may need to determine the underlying reason for the exam, if not stated explicitly, to make an accurate diagnosis. In other words, the radiologist may want to determine the reason for the exam to understand the case (e.g., urgent case, emergency case, stroke case) and/or the structures within the image that are of particular interest in the evaluation. To this end, the radiologist may search electronic medical records (EMR) to gather information and determine the reason or reasons for the exam.

The EMR may include lab notes (e.g., results of blood tests, results of genetic testing), the patient history, as well as additional medical images. Searching through the EMR may increase an amount of time taken by the radiologist to make a diagnosis. For example, the radiologist may need to look through multiple documents, determine a relevancy of each document, and then compile information from the documents. In certain instances, the radiologist may review files that may not be relevant to the underlying condition, thereby increasing the amount of time taken for review. However, it may be crucial for the radiologist to review the clinical information in order to make an accurate diagnosis. Accordingly, it is now recognized that improved systems and methods related to presenting clinical information may be useful.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a system may include a processor-based device storing and/or executing a ranking application. When executed on or in communication the processor-based device, the ranking application may determine an intent of an exam request based on one or more attributes of the exam, retrieve a plurality of documents from a database based on the determined intent, and assign a relevancy score to each of the plurality of documents based on a user profile. The ranking application may also create a list comprising the plurality of documents, wherein an order of the plurality of documents is based on the relevancy score, and populate a graphical user interface (GUI) with the list for display on the processor-based device or another processor-based device in communication with the processor-based device.

In an embodiment, a method may include determining a purpose of an exam request based on attributes of the exam request, assigning a relevancy score to each of a plurality of documents based on the determined purpose of the exam request, and adjusting the relevancy score for each of the plurality of documents based on a user profile. The method may also create a list of the plurality of documents, wherein the plurality of documents is ranked from a highest relevancy score to a lowest relevancy score and populating a graphical user interface (GUI) with the list of the plurality of documents for display on a workstation.

In an embodiment, a non-transitory, computer-readable medium comprising computer-readable code, that when executed by one or more processors, causes the one or more processors to perform operations including identifying a plurality of documents based on attributes of an exam and assigning a relevancy score to each of the plurality of documents based on a user profile. The one or more processors may also perform operations including creating a list comprising the plurality of documents based on the relevancy score, wherein the plurality of documents are ordered from a highest relevancy score to a lowest relevancy score and populating a graphical user interface (GUI) with the list for display on a workstation.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an embodiment of a workstation for displaying image data and storing or accessing clinical context documents, in accordance with aspects of the present disclosure;

FIG. 2 illustrates a graphical user interface (GUI) for display on a workstation and listing a ranked list of documents, in accordance with aspects of the present disclosure;

FIG. 3 illustrates a flow chart of an example method to adjust a ranking of the list of documents displayed on a workstation, in accordance with aspects of the present disclosure;

FIG. 4 illustrates a GUI displayed on a workstation and presenting an exam order form, in accordance with aspects of the present disclosure;

FIG. 5 illustrates a flow chart of an example method to assign a relevancy score to the clinical context documents based on an intent of the exam order form and display the list of documents on a workstation, in accordance with aspects of the present disclosure;

FIG. 6 illustrates a GUI displayed on a workstation and for displaying a clinical context document, in accordance with aspects of the present disclosure;

FIG. 7 illustrates a GUI displayed on a workstation that may be used to create a user profile, in accordance with aspects of the present disclosure;

FIG. 8 illustrates a GUI displayed on a workstation that may be used to edit the user profile, in accordance with aspects of the present disclosure;

FIG. 9 illustrates a flow chart of an example method to assign a relevancy score to the clinical context document based on the user profile and display the list of documents on a workstation, in accordance with aspects of the present disclosure;

FIG. 10 illustrates a flow chart of an example method to adjust the list of documents displayed on workstation based on a search term, in accordance with aspects of the present disclosure; and

FIG. 11 illustrates a flow chart of an example method to identify attributes of the user profile and identifying similar user profiles based on the attributes, in accordance with aspects of the present disclosure.

### DETAILED DESCRIPTION

One or more specific embodiments of the present disclosure are described above. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

When making a diagnosis, a radiologist may receive a brief statement (e.g., a simple one sentence statement, incomplete notes or phrases, and so forth) that explains a reason (e.g., purpose) for ordering an exam from a referring physician and an associated medical image or images. In certain instances, the stated reason for the exam may be vague or unclear. For example, the referring physician may state that the reason for ordering the exam is “pain.” However, the true reason for the exam may be based on the patient's symptoms, physical examination findings, and/or other information gathered by the referring physician during the patient's evaluation. For example, the exam may be ordered to identify a suspected fracture and the referring physician may order an X-ray scan to confirm a diagnosis and/or determine a location of the fracture. In another example, a patient may experience back pain and an imaging exam, such as an X-ray, CT scan, or MRI scan, may be ordered to evaluate the lumbar spine and identify a cause of the pain. As such, the radiologist may need to determine the true intent of the exam by explore imaging and past documentation (e.g., clinical, surgical, pathology notes, prior imaging reports) in order to make an accurate diagnosis.

The radiologist may have a checklist of places to search for clinical context documents in order make the diagnosis. For example, the radiologist may search in a third-party server and/or database (e.g., EPIC database for outpatient information, a Cerner PowerChart for inpatient information, a Rad Assessment for dose contrast) for clinical context documents. While the radiologist may be thorough, searching multiple servers and/or databases for information and identifying relevant documents for clinical context may increase an amount of time needed to review the image and make a diagnosis. For example, reviewing an abdominal image with clinical context may take 11 minutes, while reviewing an abdominal image without clinical context may take 4.8 minutes. In another example, reviewing a neurology image with clinical context may take 12.8 minutes, while reviewing neurology image without clinical context may take 3.8 minutes. The reading time with clinical context includes time needed to read the exam plus the time needed to search for clinical context documents, evaluate the documents for relevancy, and use the documents to determine the true intent of the ordered exam, while the read time without clinical context is the time needed to read the exam when there is no need to search for additional context because the reason for the exam is well-defined. In certain instances, the best source of information is the referring physician.

In certain instances, image reviews may be performed by artificial intelligence (AI), and the radiologist may either accept or reject an AI finding. To this end, the radiologist may use clinical context documents to determine the true intent of the ordered exam and also justify their decision to either accept or reject the AI finding. As described above, the radiologist may search through multiple data sources for a complete patient history and filter through the patient history to identify information relevant to the true intent of the ordered exam. However, searching for clinical context documents and determining the relevancy of the documents may be time-consuming and inefficient process.

Embodiments of the present disclosure are directed to systems and methods for creating a user profile and ranking a plurality of documents received from one or more data sources (e.g., EPIC database, Cerner PowerChart, Rad assessment) based on the user profile. For example, the user profile may include attributes associated with the user's workplace, personal preferences of the user, and/or attributes related to the role the user is performing. The personal preferences of the user may include interactions with a plurality of clinical context documents, such as clinical notes, pathology reports, lab values, and surgical notes. A relevancy score related to a reason (e.g., purpose) for the exam may be determined for some or all of the documents of the plurality of documents. For example, the relevancy score may be assigned based on combined aspects of user preferences, clinical context document features, exam metadata (e.g., attributes), and the like. Some or all of the plurality of documents may be ranked from most relevant to least relevant based on the relevancy score. The relevancy score may be adjusted based on the user profile to personalize the ranking (e.g., ordering, prioritization) of the plurality of documents. The adjustment is made based on the user preferences associated with the user profile. Additionally or alternatively, the relevancy score may be adjusted based on preferences of other, similar users at an institution level, a local geography level, or worldwide. In this way, the plurality of documents may be personally ranked for the user based on the user profile (or the profiles of similar users), thereby decreasing search time needed to locate relevant documents and improve quality of service by prioritizing the most relevant clinical context documents first in a list of the documents.

Embodiments of the present disclosure may utilize machine-learning routines to learn the preferences of the user and other, similar users. For example, the user may view the list of documents and edit the ranking. The system may learn the preferences of the user, such as based upon their updates or changes, and store the preferences in the user profile. Furthermore, the system may identify similar users and apply the preferences to the user. For example, the system may apply preferences of other users at the institution level, the local geography level, or worldwide. By leveraging knowledge of similar users, junior or intermediate radiologists may benefit by learning about the type of information other, similar users rely on to make decisions and/or diagnosis. In this way, training time for junior radiologists may be reduced.

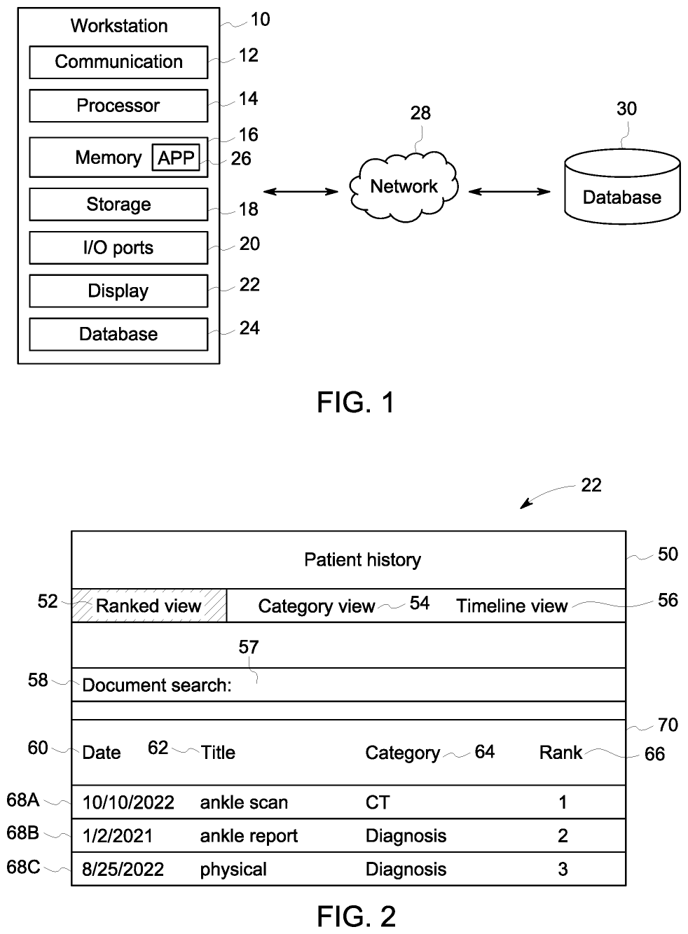

With the preceding in mind, FIG. 1 illustrates an embodiment of a workstation 10 for displaying image data and storing clinical context documents, in accordance with aspects of the present disclosure. The workstation 10 may receive raw or pre-processed image data from one or more data sources and process the signals into image data via signal processing techniques, machine-learning routines, artificial intelligence, and so on. The workstation 10 may also receive processed or pre-processed image data and perform image visualization techniques. To this end, a processor-based device, here represented as a workstation 10, may include any suitable computer device, such as a general-purpose personal computer, a laptop computer, a tablet computer, a mobile computer, and the like that includes specific computer-readable instructions in accordance with present embodiments. In an embodiment, the workstation 10, or other processor-based device, may include or communicate with a picture archiving and communications system (PACS) that may store and transmit information captured by medical imaging. For example, the information may include image data such as computed tomography (CT), X-Ray, (positron emission tomography (PET), single-photon emission computed tomography (SPECT), tomosynthesis, mammography, fluoroscopy, magnetic resonance imaging (MRI), and so on. The PACS may in turn be coupled to a remote client, radiology department information system (RIS), hospital information system (HIS) or to an internal or external network, so that others at different locations may gain access to the raw or processed image data.

The workstation 10 may include various types of components that may assist the workstation 10 in performing various types of tasks and operations. For example, the workstation 10 may include a communication component 12, a processor 14, a memory 16, a storage 18, input/output (I/O) ports 20, a display 22, a database 24, and the like. During operation, the memory 16 may store a ranking application 26 that, when executed by the processor 14, receives a user input of an exam, identify one or more clinical context documents, ranks the clinical context documents from a most relevant to a least relevant based on a user profile, and displays the list for the user. The ranking application 26 may include, access, or be updated using a machine-learning routine that may be trained based on the user profile. For example, the ranking application 26 may include a machine-learning routine that utilizes one or more machine-learning models. A machine-learning model may include attributes of the user (e.g., specialty, seniority, experience in role), preferences of the user (e.g., show most recent document at the top, show all the documents ordered by referring physician at the top, show documents most relevant to reason for exam at the top), and previous actions of the user (e.g., search terms, previous clinical context documents opened, ranking adjustments) in order to rank clinical context documents. In another example, the application 26 may implement deep learning to learn the preferences of the user over time. For example, the application 26 may include a user profile with predefined preferences as an initial point. As the user operates the workstation 10, the ranking application 26 may learn the preferences of the user, such as which clinical context documents may be relevant to the user and which documents may not be relevant. The ranking application 26 may assign a relevancy score to each clinical context document based on preferences of the user, then the ranking application 26 may create a ranked list of documents. In certain instances, the user may adjust the ranking of the documents and the application 26 may learn the adjustments. In another example, the application 26 may apply a weighting system based on the preferences of the user and create a list of documents based on the weighting system. The application 26 may assign each clinical context document a relevancy score by weighing the preferences of the user. In this way, the user may view the most relevant documents first and quickly find the clinical information needed for making a diagnosis.

The communication component 12 may be a wireless or wired communication component that may facilitate communication between the workstation 10 and various other workstations via a network, the Internet, or the like. For example, the communication component 12 may send or receive images from other workstations.

The processor 14 may be any type of computer processor or microprocessor capable of executing computer-executable code. For example, the processor 14 may be configured to receive user input, such as a type of exam, a user profile, a search term, scanning parameters, or the like. Thus, the operator may select image data for viewing on the workstation 10, search for clinical context documents, adjust the ranking of a list of clinical context documents, and/or otherwise operate the workstation 10. Further, the processor 14 may be communicatively coupled to other output devices, which may include standard or special purpose computer monitors associated with the processor 14. One or more workstations 10 may be communicatively coupled for requesting examinations, viewing images, sending images, storing images, and so forth. In general, displays, printers, workstations, and similar devices supplied with or within the system may be local to the data acquisition components, or may be remote from these components, such as elsewhere within an institution (e.g., teaching hospital, private clinic, teleradiology practice), or in an entirely different location, linked to the workstation 10 via one or more configurable networks, such as the Internet, virtual private networks, and so forth. The processor 14 may also include multiple processors that may perform the operations described below.

The memory 16 and the storage 18 may be any suitable articles of manufacture that can serve as media to store processor-executable code, data, or the like. These articles of manufacture may represent computer-readable media (e.g., any suitable form of short-term memory or long-term storage) that may store the processor-executable code used by the processor 14 to perform the presently disclosed techniques. As used herein, applications may include any suitable computer software or program that may be installed onto the workstation 10 and executed by the processor 14. The memory 16 and the storage 18 may represent non-transitory (e.g., physical) computer-readable media (e.g., any suitable form of memory or storage) that may store the processor-executable code used by the processor 14 to perform various techniques described herein. For example, the memory 16 may include machine-learning routines, machine-learning models, and/or deep learning routines that may be utilized by the ranking application 26 to rank clinical context documents based on a user profile.

The memory 16 may store the ranking application 26, such as for execution by the processor 14. The application 26, when executed, may receive user input to perform an exam, identify an intent of the exam, receive or retrieve a user profile, identify one or more attributes of the user profile, and rank a plurality of clinical context documents based on the user profile and/or the intent of the exam. That is, the application 26 may determine a relevancy score for each clinical document of interest based on the preferences of the user and/or the intent of the exam. In certain instances, the application 26 may determine a relevancy score for each clinical context document based on the intent of the exam and adjust the relevancy score based on the user profile. Additionally or alternatively, the application 26 may determine a relevancy score based on preferences of users with similar profiles as the operator. Based on the relevancy score, the application 26 may rank the clinical context documents to streamline the review process for the operator and reduce time needed to make a diagnosis. The application 26 may populate a graphical user interface (GUI) with the list of documents. In this way, the application 26 may display the clinical context documents from most relevant to least relevant for the user, thus reducing an amount of time needed to find clinical information and make a diagnosis.

Returning to the workstation 10, the I/O ports 20 may be interfaces that may couple to other peripheral components such as input devices (e.g., keyboard, mouse), sensors, input/output (I/O) modules, and the like. The display 22 may operate as a human machine interface (HMI) to depict visualizations associated with software or executable code being processed by the processor 14. For example, the display 22 may display the GUI with the list of clinical context documents. In another example, the display 22 may display a GUI with the user profile and receive user input to adjust an attribute of the user profile. In one embodiment, the display 22 may be a touch display capable of receiving inputs from a user of the workstation 10. The display 22 may be any suitable type of display, such as a liquid crystal display (LCD), plasma display, or an organic light emitting diode (OLED) display, for example. Additionally, in one embodiment, the display 22 may be provided in conjunction with a touch-sensitive mechanism (e.g., a touch screen) that may function as part of a control interface for the workstation 10.

The database 24 may locally store image data, clinical context documents, and/or user profiles within the workstation 10. In an example, exam metadata may be stored within the database 24, and each exam metadata may be tagged with an embedding. For example, the database 24 may store clinical context documents received from the data sources for review. Each clinical context document may be represented using (e.g., associated with) an embedding (e.g., numerical vector in a low-dimensional space). The embedding may capture a meaning or a context of the words and phrases used to facilitate the computation of similarity between documents. In another example, the database 24 may store the user profile, including data regarding adjustments made by the user, historical search data (e.g., search terms, exam types), historical ranking data (e.g., adjustments), and the like. The user profile may also be represented by an embedding. Additionally or alternatively, the database 24 may store a plurality of user profiles, the historical search data for multiple users, historical ranking data for multiple users, and the like. As further described herein, the application 26 may compute similarity between clinical context documents, exam metadata, and/or user profiles using the embeddings.

In an instance, the database 24 may store the machine-learning model used by the application 26 to create the list of clinical context documents. In certain instances, the database 24 may store multiple machine-learning models utilized by the application 26 to create the list of documents. For example, a first machine-learning model may store the preferences of the user, a second machine-learning model may store a comparison between one or more user profiles, a third machine-learning model may store attributes of the clinical context documents, a fourth machine-learning model may store attributes of the exam, and so on. The application 26 may utilize one or more machine-learning models to determine the final relevancy score of the clinical context documents to create the list of documents. In this way, the application 26 is creating a personalized list of documents for the user to streamline the review process and decrease time needed to make a diagnosis. Although the database 24 is illustrated as part of the workstation 10, in an embodiment, the database 24 may be a cloud server or a remote server that is communicatively coupled to the workstation 10.

It should be noted that the workstation 10 should not be limited to include the components described above. Instead, the components described above with regard to the workstation 10 are examples, and the workstation 10 may include additional or fewer components relative to the illustrated embodiment. For example, the processor 14 and the memory 16 may be provided collectively within the workstation 10.

In certain embodiments, the workstation 10 may be communicatively coupled to a network 28, which may include collections of workstations, the Internet, an Intranet system, or the like. The network 28 may facilitate communication between the workstation 10 and various other data sources. For example, the network 28 may facilitate communication between the workstation 10 located on the surgery floor and a workstation 10 located on the radiology floor. In another example, the network 28 may facilitate communication between the workstation 10 and a third-party server and/or database (e.g., EPIC database, a Cerner PowerChart, a Rad Assessment) to receive clinical context documents. In another example, the network 28 may facilitate communication between the workstation 10 and a database 30. The database 30 may be a cloud database or a remote server. As described herein, the database 30 may store the user profile, image data, clinical context documents, the machine-learning model, associated embeddings, and the like.

FIG. 2 illustrates a graphical user interface (GUI) 50 displaying the list of clinical context documents, in accordance with aspects of the present disclosure. For example, the GUI 50 may be displayed on the display 22 of the workstation 10 and may provide user selectable options (here “tabs” determining the view) configured to receive a user input. The user input may be a selection to view the list of clinical context documents in a “RANKED VIEW” 52, a “CATEGORY VIEW” 54, or a “TIMELINE VIEW” 56. The ranked view 52 may allow the user to view the documents based on a rank (e.g., relevancy score). For example, the application 26 may assign a relevancy score to each relevant clinical context document and rank the documents from a highest relevancy score to a lowest relevancy score. The application 26 may determine the relevancy scores based on the machine-learning models, the user profile, the intent of the exam, the preferences of other similar users, and the like. The category view 54 allows the user to view the clinical context documents based on a category selection (e.g., the type of or contents of each record or document such as clinical notes, pathology reports, surgery notes, lab results). If applicable, a category may be filtered by Body Part under consideration. The application 26 may assign a relevancy score to each clinical context document, then adjust the relevancy score based on the Body Part selection. For example, the user may select the Body Part as “head” and the application 26 may prioritize clinical context documents related to the head. The timeline view 56 allows the user to view the documents from a most recent document to a least recent document or vice versa. For example, the application 26 may identify a date or a time the document was created and/or entered into the workstation 10 and use the date to create the list of documents.

In certain instances, the GUI 50 may receive a search term. The GUI 50 may include search bar 57 for the user to enter a search term 58. As illustrated, the search bar 57 states, “DOCUMENT SEARCH:” and the user may enter the search term 58. In certain instances, the application 26 may populate the GUI 50 with the list of clinical context documents and the user may readjust the list by entering the search term 58. For example, the application 26 may adjust the relevancy score based on the search term 58 and repopulate the GUI 50 with the list of clinical context documents. That is, the application 26 may reorder the documents within the list of clinical context documents based on the adjusted relevancy score. As such, the user may be more specific in identifying relevant clinical context documents. Additionally, the application 26 may save the search term in the user profile to learn the preferences of the user.

Returning the list of clinical context documents, the application 26 may populate the GUI 50 with attributes of the documents. As illustrated, the GUI 50 may display a “DATE” 60, a “TITLE” 62, a “CATEGORY” 64, and/or a “RANK” 66. As such, the user may quickly understand the attributes of the document and determine if the document may be relevant. For example, the GUI 50 may display a first clinical context document 68A, a second clinical context document 68B, and a third clinical context document 68C (collectively referred to as “clinical context documents 68”). The first clinical context document 68A may be a CT ankle scan saved on Oct. 10, 2022, the second clinical context document 68B may be an ankle report saved on Jan. 2, 2021, and the third clinical context document 68C may be an annual physical saved on Aug. 25, 2022. The application 26 may rank the clinical context documents 68 based on a relevancy score and display the documents 68 as a list of documents 70. As indicated by the list of documents 70, the ranking application 26 may determine the first clinical context document 68A to be more relevant in comparison to the second clinical context document 68B and the third clinical context document 68C based on the user profile 78 and/or the reason for the exam. Additionally or alternatively, the ranking application 26 may determine the first clinical context document 68A to be more relevant in comparison to the second clinical context document 68B and/or of the third document 68C based on other similar user profiles and the preferences of the other users. In this way, the ranking application 26 may present the list of clinical context documents 70 based on relevance to the user, thereby decreasing an amount of time needed for the user search for clinical context documents 68 and make a diagnosis.

The user may edit the ranking of the list of documents 70 using the “RANK” option 66. For example, the user may adjust the ranking of the first clinical context document 68A by user input, such as indicating the first clinical context document 68A should be ranked third. In another example, the user may drag the second clinical context document 68B above the first clinical context document 68A. As such, the ranking of the first clinical context document 68A may be adjusted (e.g., reordered) from first to second while the ranking of the second clinical context document 68B may be adjusted from second to first. The application 26 may learn (e.g., via machine-learning routines, deep learning) adjustments made by the user and store the adjustments as user preferences. Additionally or alternatively, the application 26 may adjust the machine-learning model based on the user inputs (e.g., adjustments). In this way, the application 26 may personalize the list of clinical context documents 70 based on the preferences of the user.

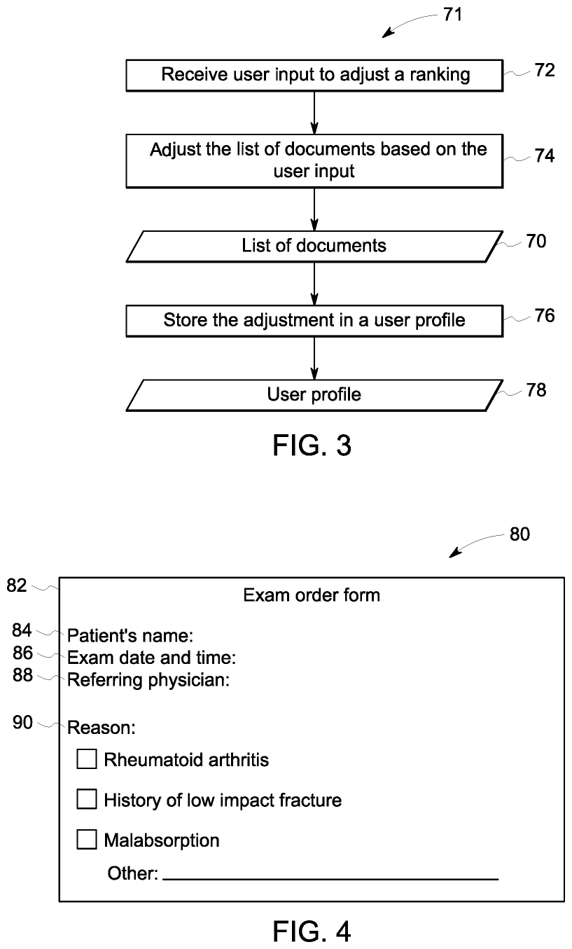

FIG. 3 illustrates a flow chart of an example method 71 for adjusting the list of documents 70 and storing the adjustments in the user profile 78, in accordance with aspects of the present embodiment. One or more steps of the illustrated method 71 may be performed in a different order from the order depicted in FIG. 3 and/or simultaneously (e.g., in parallel) by the application 26.

As described herein, the ranking application 26 may populate the GUI 50 with the list of documents 70 to streamline the process for making a diagnosis. In certain instances, the user may adjust the ranking of one or more clinical context documents 68 based on personal preferences. At block 72, the application 26 may receive user input to adjust a ranking. For example, the user input may be adjusting a ranking of the first clinical context document 68A to be third in the list of documents 70. In another example, the user input may be adjusting the ranking of the third clinical context document 68C to be second in the list of documents. Still in another example, the user input may be indicating that the second clinical context document 68B may not be relevant to the exam. As such, the application 26 may learn the preferences of the user.

At block 74, the ranking application 26 may adjust the list of documents 70 based on the user input. The ranking application 26 may dynamically repopulate the GUI 50 with an adjusted list of documents 70 based on the adjustment. For example, the adjusted list of documents 70 may be the third clinical context document 68C, followed by the second clinical context document 68B, and followed by the first clinical context document 68A.

At block 76, the ranking application 26 may store the adjustment in a user profile. The ranking application 26 may analyze the attributes of each clinical context document 68 and identify a preference of the user. For example, the user may prefer to first review a physical report (e.g., third clinical context document 68C) to gain a general understanding of the patient then review more specific information (e.g., first clinical context document 68A, second clinical context document 68B). By storing the adjustments of the user, the ranking application 26 may learn the user preferences over time. As such, recommendations may become more granular based on the user preferences as the user preferences are updated or adapted over time.

FIG. 4 illustrates a GUI 80 for analyzing an exam order form 82 to determine an intent of the exam, in accordance with aspects of the present disclosure. As described herein, the intent of the exam is used to described a clinical indication or a purpose of the exam. The purpose of the exam can help users (e.g., radiologist) understand the context in which the exam was performed and to focus on the specific areas of concern. The purpose of the exam may be based on the patient's symptoms, physical examination findings, or other information gathered during the patient's evaluation. For example, if a patient is experiencing chest pain, a chest X-ray or CT scan of the chest may be ordered to evaluate the heart, lungs, and/or other structures of the patient's chest for signs of a problem, such as pneumonia, a pulmonary embolism, or a heart attack. For suspected pneumonia, the referring physician may order a chest x-ray scan to confirm a diagnosis.

Returning to the ranking application 26 may analyze the exam order form 82 to determine the true intent (e.g., reason, purpose) for the study, examination, or imaging session. Based on the intent, the ranking application 26 may determine the relevancy score for the clinical context documents 68.

As illustrated, the exam attributes (e.g., exam metadata) may include a patient name (or identifier) 84, an exam date and time 86, a referring physician 88, and a reason 90. The patient name (or identifier) 84 may include the name of or identifying information for the patient and the application 26 may utilize the patient name 84 or identifying information to identify clinical context documents associated with the exam. The exam date and time 86 may be the time point in which the exam occurred. The application 26 may utilize the exam date and time 86 to determine a relevancy of clinical context documents. For example, clinical context documents closer in date to the exam order may be more relevant in comparison to clinical context documents farther in date to the exam order. The referring physician 88 may be the physician ordering the exam. The reason 90 may provide an indication of the true intent for ordering the exam. For example, the referring physician 88 may check a box associated with Rheumatoid Arthritis. In another example, the referring physician 88 may write the reason in the category ‘other.’ The indication provided in the reason 90 may be utilized by the user and/or the application 26 to determine the true intent of the exam.

Additionally, the exam attributes may include patient demographics, a primary physician, the primary physician's institution, a reason for exam, a modality, a protocol, and so on. The patient demographics may include patient identifying information, such as a name, patient or hospital number, a date of birth, an address, sex, insurance information, and the like. The referring physician may be a non-radiologist physician who sends a patient to a specialist for certain medical services. For example, the referring physician may be a family doctor sending the patient to get an X-ray. Then, the user (e.g., the radiologist) may receive the X-ray image data and make a diagnosis based on the X-ray imagery. The primary physician may be a day-to-day healthcare provider for the patient. In certain instances, the primary physician and the referring physician may be the same person. For example, the primary physician may execute the exam order form 82 for the patient to see a specialist. The exam date and the exam time may the date and time schedule for the exam. The modality may include a type of image data, such as CT, MRI, PET, X-ray, nuclear medicine imaging, and the like. For example, the referring physician may order a CT exam. The protocol may be a description of how the exam should be conducted. For example, the protocol may include a contrast dose, a region for imaging, a number of passes over the patient's body, and the like. As such, the application 26 may utilize the exam attributes to determine an intent of the exam as well as to determine a relevancy score for the clinical context documents 68.

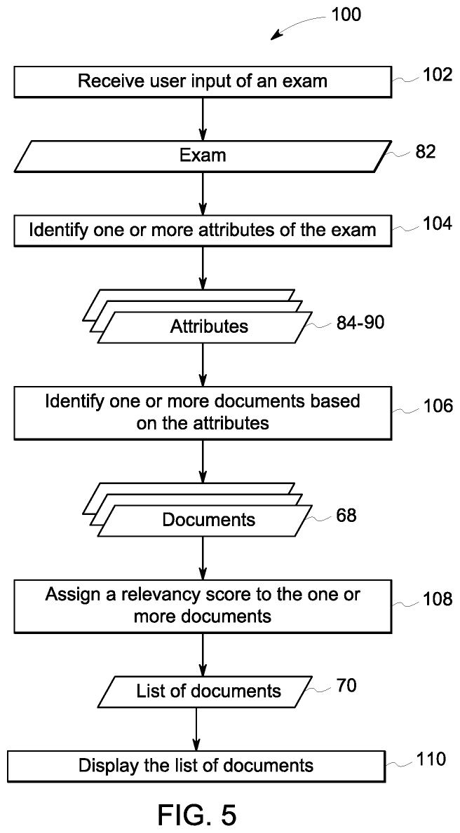

FIG. 5 illustrates a flow chart of an example method 100 for the ranking application 26 to identify a relevancy score for a clinical context document based on the exam order form 82, in accordance with aspects of the present disclosure. One or more steps of the illustrated method 100 may be performed in a different order from the order depicted in FIG. 5 and/or simultaneously (e.g., in parallel) by the application 26.

By way of example, the application 26 may assign a relevancy score to each clinical context document based on Equation 1, described below:

$$Relevancy Score = f ( {v}_{feat} , {S}_{exam , exams \_ pool} , {S}_{user , profiles \_ pool} , {S}_{doc , docs \_ pool} ) \qquad ( 1 )$$

The application 26 may utilize embeddings associated with each input to compute a similarity. The application 26 may combine aspects of user preferences, clinical context document features, and/or exam metadata to determine the relevancy score. For each user profile, the application 26 may compute similarity to a target pool of user profiles (S_{user,profiles_pool}). For each exam, the application 26 may compute a similarity to a target pool of prior exams reviewed by the user (S_{exam,exams_pool}). For each clinical context document, the application 26 may compute a similarity to a target pool of prior documents labeled as relevant clinical context documents by the user (S_{doc,docs_pool}). Additionally or alternatively, a value of a feature (v_{feat}) from a feature set provided as a result of analyzing the user profile for the user preferences, such as displaying most recent document at the top, displaying documents most relevant to search criteria at the top, weight given to each ranking criteria, etc. Thus, the relevancy score may be determined as a function of an identified feature and similarity scores for identified user, exam, and clinical context documents.

With the preceding in mind, at block 102, the ranking application 26 may receive user input of an exam. For example, the ranking application 26 may receive and analyze the exam order form 82. In another example, the ranking application 26 may receive user input of the exam, including one or more attributes of the exam. Still in another example, the ranking application 26 may compute similarity to a target pool of prior exams interpreted by the user.

At block 104, the ranking application 26 may identify one or more attributes of the exam. For example, the ranking application 26 may identify attributes 84-90 of the exam order form 82. As described with respect to FIG. 4, the exam attributes may include patient demographics, a referring physician's name, a referring physician's institution, an exam date, an exam time, a reason for the exam, an imaging modality, an imaging protocol, or the like. The application 26 may discern an intent for the exam based on the attributes 84-90 of the exam.

At block 106, the ranking application 26 may identify one or more clinical context documents 68 based on the exam attributes 84-90. As described with respect to FIG. 5, the application 26 may identify one or more attributes of the clinical context document 68. The ranking application 26 may identify matching (e.g., overlapping) attributes between the exam order form 82 and the clinical context documents 68 to identify relevant documents 68. For example, the ranking application 26 may search the database 24 and 30 with the patient's name 84 and date of birth to identify the patient's history. In another example, the ranking application 26 may use the patient's name 84 and the name of the referring physician 88 to identify clinical context documents 68 associated with the patient. In certain instances, the application 26 may assign a higher relevancy score for a greater number of matching attributes between the exam and a clinical context documents in comparison to a smaller number of matching attributes. In other instances, the application 26 may compute a similarity to a target pool of prior documents labeled as relevant clinical context documents by the user.

At block 108, the ranking application 26 may assign a relevancy score to each of the clinical context documents 68. The relevancy score may be indicative of how relevant or useful the document might be to the user. Indeed, a higher relevancy score may indicate that the document is more useful or preferred by the operator and a lower relevancy score may indicate that the document is less useful. For example, the ranking application 26 may determine the intent of the exam based on the attributes 84-90 of the exam order form 82. Then, the ranking application 26 may compare the attributes of the clinical context documents 68 to the intent (e.g., purpose) to determine the relevancy score. For example, the ranking application 26 may determine the intent of the exam is to determine a size of a tumor in the brain. The ranking application 26 may prioritize clinical context documents 68 associated with the head or the brain of the patient. That is, the ranking application 26 may assign a higher relevancy score to documents 68 associated with the head or brain in comparison to documents 68 associated with the foot or the arm. For example, a ranked ordering of the list of clinical context documents may be from a highest relevancy score to a lowest relevancy score. In another example, the ranking application 26 may determine the intent is related to a pain in the arm. As such, the ranking application 26 may assign a relevancy score to clinical context documents 68 related to the arm higher in comparison to documents not associated with the arm. Still in another example, the ranking application 26 may assign a higher relevancy score based on a number of matching attributes 84-90 between the exam order form 82 and the clinical context document 68.

The ranking application 26 may adjust the relevancy score based on the user profile 78. For example, the ranking application 26 may account for user preferences and adjust the relevancy score. The ranking application 26 may create a list of clinical context documents 70 based on the relevancy score. For example, the ranking application 26 may prioritize (e.g., ranked ordering, order, organize) the clinical context documents 68 from a highest relevancy score to a lowest relevancy score. In this way, the ranking application 26 may create a personalized list for the user.

At block 110, the ranking application 26 may display the list of documents 70. That is, the ranking application 26 may populate a GUI (e.g., GUI 40 described with respect to FIG. 2) with the list of clinical context documents 70 ranked from a highest relevancy score to a lowest relevancy score. By providing a ranked list of clinical context documents, the user may save time by skipping the search for relevant documents. Rather, the user may use the ranked list of documents 70 to quickly and efficiently gather additional information related to the exam and to make a diagnosis.

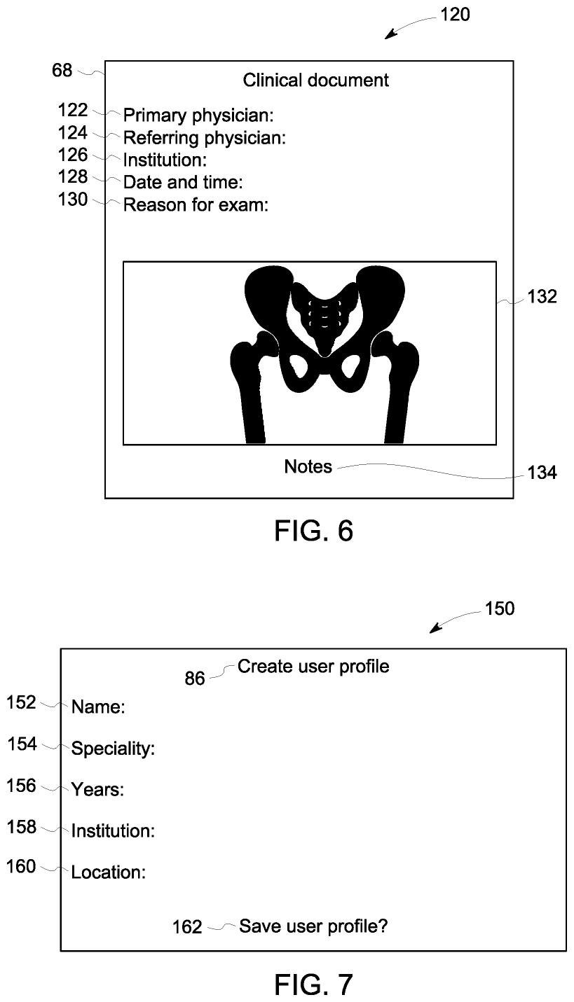

FIG. 6 illustrates a GUI 120 for analyzing a clinical context document 68 to determine a relevancy score, in accordance with aspects of the present disclosure. For example, the application 26 may perform operations, such as retrieval and computing relevancy, on clinical context documents 68. The application 26 may analyze each clinical context document 68 and add metadata to facilitate the comparison between the document 68 and the intent of the exam. Additionally or alternatively, the application 26 may analyze the document 68 in light of the user profile and/or user preferences identified from the user profile. In particular, the application 26 may adjust a relevancy score based on the preferences of the user and/or the attributes of the user within the user profile. Additionally or alternatively, the application 26 may compare the document to the preferences of other similar users. In this way, the application 26 may determine a relevancy score for the document that is useful to the user based on the user's explicit preferences or the preferences of those considered similar to the user.

As illustrated, the clinical context document 68 may include information on a primary physician 122, a referring physician 124, the referring physician's institution 126, a date and time 128, a reason for the exam 130, an image 132, and notes 134. As described herein, the primary physician 122 may be a day-to-day physician of the patient and the referring physician 124 may be the physician ordering the exam. The clinical context document 68 may also include the image 132 taken during the exam. The application 26 may use image processing techniques to determine type of image data, a region imaged, annotations on the image, and the like. The clinical context document 68 may also include notes 134 by the reviewing physician. For example, the notes 134 may include a diagnosis, surgical notes, clinical notes, and the like. The application 26 may analyze the attributes 122-134 of the clinical context document 68 and use the attributes to determine a relevancy score.

Additionally or alternatively, the clinical context document attributes may include a category (e.g., clinical notes, lab values, pathology reports, surgical notes, scanned documents), a source of the document (e.g., EPIC, Cerner PowerChart, Rad Assessment, EMR), a document quality (e.g., based on level of detail provided), a document content, and so on. The source of the document may indicate a status of the patient at the time of the exam. As described herein, the EPIC database may be searched for outpatient information, the Cerner PowerChart may be searched for inpatient information, the Rad Assessment may be searched for dose contrast, and the EMR may be searched for outpatient information. In certain instances, the application 26 may utilize natural language processing (NLP) to analyze the clinical context document 68 for the document quality and/or the document attributes. For example, certain notes within the clinical context document 68 may not be relevant to the user based on the user profile or the reason for the ordered exam. In another example, the clinical context document 68 may include a high level of detail indicating the reason for the exam, the previous patient history, the diagnosis and so forth.

By way of example, the application 26 may analyze the clinical context document 68 and identify that the same referring physician may recommend an MRI as the current exam. However, the referring physician may have recommended the MRI 5 years ago, as indicated by the clinical context document. Since the recommendation was made 5 years ago, the clinical context document may not be relevant to the radiologist reviewing the exam who is only interested in using recent prior information for context. As such, the application may assign a lower relevancy score in comparison to a clinical context document from 5 days ago that may relate to whether or not the notes are relevant.

FIG. 7 illustrates a GUI 150 for creating a user profile 78, in accordance with aspects of the present disclosure. As described herein, the application 26 may utilize a user profile 78 with one or more preferences as an initial point. The user profile 78 may include workplace attributes, personal preference attributes, and/or role attributes. The workplace attributes may account for location factors that may influence the preferences of the user. For example, an institution may train their physicians in a manner different from another institution. The workplace attributes may include an institution name, an institution location, an institution type, and/or an institution size.

The personal preferences attributes may account for individual factors of the user. For example, personal preference attributes may include ranking a most recent document as relevant or highly relevant, ranking a latest of the most frequent documents as relevant or highly relevant, documents ordered by the referring physician as relevant or highly relevant, documents most relevant to reason for exam as relevant or highly relevant, documents most relevant to search criteria as relevant or highly relevant, documents manually ranked by the user with respect to reason for exam as relevant or highly relevant, and/or a weight given to each ranking criteria. For example, the user may prefer to see the most recent document at the top of the list of relevant documents.

The role attributes may account for factors related to the position or role in which the user performs their work. For example, role attributes may include seniority (e.g., resident, fellow, attending), a specialty (e.g., neuroradiology, abdominal imaging, pediatrics, women's health), a modality (e.g., CT, MR, PET), a body part (e.g., head, chest, breast, abdomen), an experience in role in years, a total experience in years, and/or an activity type (e.g., teaching, peer review, supervising). For example, the application 26 may leverage preferences of more senior users for more junior users to improve training and/or decrease training time. In another example, the application 26 may apply a preference of a fellow radiologist to a resident radiologist. Still in another example, the application 26 may apply the preference of a fellow radiologist to other fellow radiologist since the radiologists at each seniority level may have different preferences.

As illustrated, the GUI 150 includes user attributes, such as a user's name 152, a specialty 154, a number of years 156, an institution 158, and/or a location 160. The GUI 150 may also include a button 162 that saves the user profile in response to user input. In certain instances, the name 152 may be the name of the user and used by the application 26 to identify the user profile. In another instance, the name 152 may be a username assigned to the user, such as an email address. By way of example, the specialty 154 may be a field of radiology the user primarily practices in. In another example, the specialty 154 may be an area of medicine in which the user practices. The number of years 156 may be a number of years the user has been practicing medicine. In certain instances, the number of years 156 may be a seniority level, such as resident, fellow, or attending. The location 160 may be a geographical location where the user is located. Within the user profile 78, the application 26 may store preferences based on the nature of the image and/or the role being performed.

As the application 26 continues to learn, the recommendations (e.g., list of documents) may get more granular based on the clinical context documents 68 searched for. For example, the user may frequently use a search term and the application 26 may save the search term to the user profile. When the user performs a subsequent search, the application 26 may predict the user's preferences and recommend similar clinical context documents based on the frequently used search term, thus saving time. In another example, the user (e.g., radiologist) may treat a patient for cancer and may only review clinical context documents 68 at the time of diagnosis and at present time. The application 26 may learn this preference over time and present the initial clinical context documents and current clinical context documents for the user when the exam is related to cancer. In this way, the application 26 may learn the preferences of the user over time.

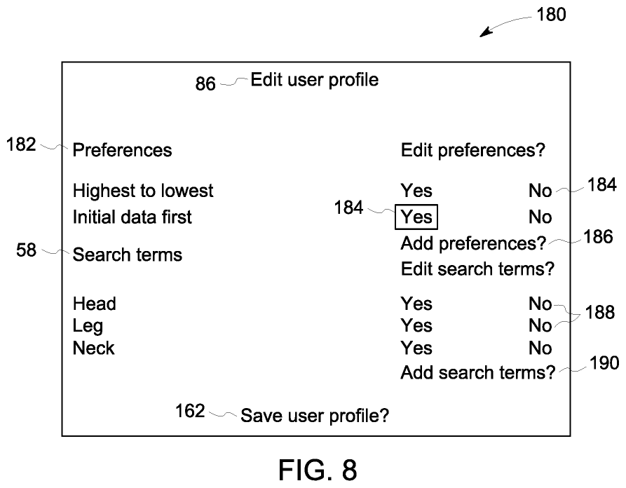

FIG. 8 illustrates a GUI 180 for editing (e.g., modifying) the user profile 78, in accordance with embodiments of the present disclosure. For example, the user may modify one or more attributes or preferences stored within the user profile 78 and the application 26 may update the user profile based on such user inputs.

As illustrated, the GUI 180 may include preferences 182 and search terms 58 identified by the application 26. The preferences 182 and the search terms 58 may be collected over time and stored with the user profile 78. The preferences 182 may include presenting the list from a highest relevancy score to a lowest relevancy score and presenting initial data at a top of the list. The user may edit the preferences 182 by selecting an edit button 184. For example, the user may not want the initial data (e.g., at time of diagnosis) presented first. As such, the user may delete the preference 182. In another example, the user may want the most current documents presented first. The user may add the preference using an “add preferences” button 186 and the application 26 may store the preference in response to the user input.

Additionally or alternatively, the user may modify one or more search terms 58. As described with respect to FIG. 2, the search term 58 may be entered to narrow the list of clinical context documents 68. For example, the application 26 may identify one or more clinical context documents 68 based on the attributes 84-90 of the exam order form 82. The user may input a search term 58 of the GUI 50 to decrease a number of clinical context documents 68 presented in the list of clinical context documents 70. The application 26 may store the search terms 58 in the user profile 78 as a user preference. For example, the search terms 58 may include anatomical regions (e.g., head, leg, and neck, and so forth). The search terms 58 may also include a referring physician's name, the referring physician's institution, a primary physician's name, the primary physician's institution, an exam date, and exam time, a type of image, and so on. The user may change specialties or no longer use a search term 58. As such, the user may edit the search terms 58 by using the buttons 188. For example, the user may delete a search term or modify a search term. Further, the user may add a search term 58 to the list by using the “add search terms” button 190.

Additionally or alternatively, the GUI 180 may include the user attributes 152-160 described with respect to FIG. 7 for modification. For example, as the user progresses in their career their seniority level may change. As such, the user may edit the roles attributes category. In another example, the user may switch institutions. As such, the user may edit the workplace attributes.

In certain instances, the application 26 may remove a search term 58 after a threshold period of time if the user does not use the search term. In this way, the application 26 may learn the preferences of the user. Additionally or alternatively, as the user progresses in their career, their preferences 182 may change. As such, the application 26 may modify (e.g., remove, adjust, add) the preferences 182 of the user over time. For example, the user may advance from a resident to a fellow. The application 26 may identify preferences of fellows that may be different from preferences of residents and apply the preferences to the user's profile. Still in another example, the application 26 may adjust preferences 182 of the user based on user feedback (e.g., adjusting the list of documents).

The application 26 may learn user preferences by analyzing search results and user feedback. The application 26 may also learn what constitutes a similar profile and similar documents. The application 26 may also learn the preferred combination of ranking criteria per user. In this way, the application 26 may generate personalized lists for the user.

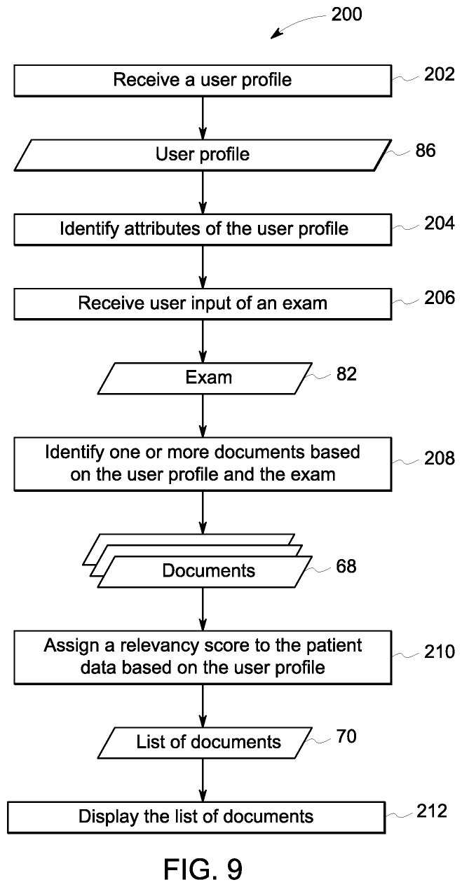

FIG. 9 illustrates a flow chart of an example method 200 for the ranking application 26 to generate the list of clinical context documents 70 based on the user profile 78, in accordance with aspects of the present disclosure. One or more steps of the illustrated method 200 may be performed in a different order from the order depicted in FIG. 9 and/or simultaneously (e.g., in parallel) by the application 26.

At block 202, the ranking application 26 may receive a user profile 78. For example, the user may log into the workstation 10 with a user ID and a password. The ranking application 26 may identify the user profile 78 based on the user ID. In another example, the user may scan a badge to operate the workstation 10 and the ranking application 26 may identify the user profile 78 based on information received by scanning the badge.

At block 204, the ranking application 26 may identify attributes of the user profile 78. For example, the application 26 may identify the workplace attributes, the personal preference attributes, and/or the role attributes within the user profile 78. In another example, the application 26 may identify the preferences 182 and/or the search terms 58.

At block 206, the ranking application 26 may receive user input indicative of an exam, similar to block 102 described with respect to FIG. 5. The ranking application 26 may identify one or more attributes 84-90 of the exam. At block 208, the ranking application 26 may identify one or more clinical context documents 68 based on the user profile 78 and the exam. For example, the application 26 may identify clinical context documents 68 associated with the exam based on a name of the patient. Then, the ranking application 26 may identify one or more clinical context documents 68 based on the user profile 78. For example, the application 26 may learn that the user does not want clinical context documents more than ten years old. As such, the application 26 may remove clinical context documents more than ten years old. In another example, the application 26 may learn that the user likes annual physical reports. As such, the application 26 may retrieve all annual physicals of the patient. In this way, the application 26 may collect all relevant clinical context documents for the user.

At block 210, the ranking application 26 may assign a relevancy score to each of the collected documents based on the user profile. The ranking application 26 may compare attributes of the clinical context documents 68 with the preferences 182 of the user to determine the relevancy score. For example, the user may want documents ordered by referring physician and the documents with the same referring physician as the exam at the top of the list. As such, the application 26 may assign clinical context documents 68 with the same referring physician as the ordered exam a higher relevancy score in comparison to clinical context documents with different referring physicians. Further, the user may focus on image data related to arms. As such, the application 26 may rank image data related to arms and the same referring physician higher in comparison to image data related to other body parts and a different referring physician. Based on the relevancy score, the application 26 may create the list of clinical context documents 70. That is, the application 26 may rank the clinical context documents 68 from a highest relevancy score to a lowest relevancy score to make the list of documents 70.

At block 212, the ranking application 26 may display the list of documents 70, similar to block 110 described with respect to FIG. 5. In this way, the application 26 may prioritize presenting the clinical context documents 68 with a highest relevancy score to the user. In other words, the application 26 may present a most relevant clinical context document to the user, thus saving the user time that may be spent searching for documents. In this way, turn-around time may decrease and user efficiency may increase.

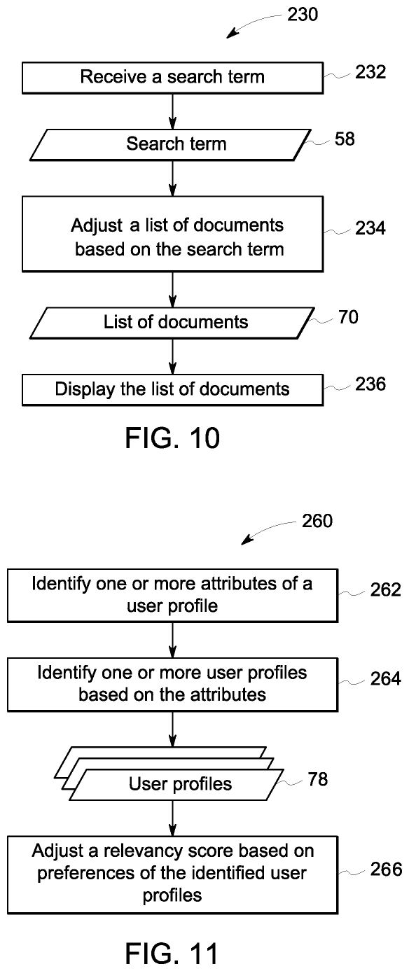

FIG. 10 illustrates a flow chart of an example method 230 of the application 26 adjusting the list of clinical context documents 70 based on a search term 58, in accordance with aspects of the present disclosure. One or more steps of the illustrated method 230 may be performed in a different order from the order depicted in FIG. 10 and/or simultaneously (e.g., in parallel) by the application 26.

The application 26 may create the list of documents 70 based on the intent of the exam and the user profile 78. In certain instances, the user may narrow the list of relevant documents based on a search term 58. As described with respect to FIG. 2, the user may input a search term 58 into the search bar 57 of the GUI 50. The ranking application 26 may receive the search term 58 at block 232. For example, the user may enter ‘head’ as the search term 58. In another exam, the user may enter the name of a referring physician as the search term 58. As such, the list of clinical context documents 70 may be adjusted based on the search term 58.

At block 234, the ranking application 26 may adjust the list of documents 70 based on the search term 58 at block 234. For example, the ranking application 26 may increase the relevancy score for clinical context documents 68 related to the search term and decrease the relevancy score for clinical context documents 68 not related to the search term. In another example, the ranking application 26 may assign new relevancy scores to each clinical context document 68. Then, the application 26 may adjust the ranking of the documents within the list of clinical context documents 70. In certain instances, the application 26 may create a new list of clinical context documents 70 based on the relevancy score. At block 236, the ranking application 26 may display the list of documents, similar to block 110 described with respect to FIG. 5.

FIG. 11 illustrates a flow chart of an example method 260 for training the ranking application 26 to learn user profiles and adjust a relevancy score, in accordance with aspects of the present disclosure. One or more steps of the illustrated method 260 may be performed in a different order from the order depicted in FIG. 11 and/or simultaneously (e.g., in parallel) by the application 26.

At block 262, the ranking application 26 may identify one or more attributes of a user profile 78. For example, the ranking application 26 may identify the workplace attributes and/or the role attributes of the user profile.

At block 264, the application 26 may identify one or more user profiles based on the attributes. For example, the application 26 may weigh user profiles within a same institution higher than user profiles at different institutions. Users trained within a same institution may have similar cultures and/or training styles which may indicate similar user preferences. In other words, users at the same institution may have similar preferences due to training and/or institution policies. In another example the ranking application 26 may identify a total experience in years since users at similar experience levels may have similar preferences. The application 26 may weigh user profiles with similar or greater seniority higher than user profiles with different or lesser seniority. Still in another example, the ranking application 26 may identify personal preference attributes and compare the attributes to other user profiles. For example, the application 26 may compute similarity of retrieved clinical context documents with documents retrieved by users with similar profiles. The application 26 may learn similarities between user profiles over time. In this way, the application 26 may learn the preferred combination of ranking the clinical context documents per user and for other similar users.

At block 266, the application 26 may adjust a relevancy score based on the identified user profiles. As described herein, the application 26 may create the list of documents 70 based on the user profile. In certain instances, the application 26 may adjust the relevancy score based on the preferences of the identified user profiles. For example, the application 26 may adjust the relevancy score for a junior user based on preferences of a senior user. In another example, the application 26 may adjust the relevancy score for a user based on preferences of another similar user to predict the user's preferences. As such, the application 26 may create personalized lists of clinical context documents for the user, thereby reducing an amount of time needed to make a diagnosis, streamlining the review process, and improving quality of service delivered by the user.

Technical effects of the disclosed embodiments include providing systems and methods for determining an intent of an exam, identifying clinical context documents, assigning a score to each of the clinical context documents, and ranking the clinical context documents in a list to be presented to a physician in reviewing an exam and/or making a diagnosis. The automation of determining an intent of an exam and identifying clinical context documents may decrease an amount of time the physician needs to find relevant clinical context documents and also increase the use of clinical context information by physicians. Furthermore, ranking the clinical context documents into the list enables the physician to quickly identify relevant patient history, efficiently review the patient history, and make the diagnosis. Furthermore, decreasing the amount of time needed to find relevant clinical documents may increase the use of clinical context information, thereby improving quality delivered by physicians. In this way, the physician may decrease time needed to make the diagnosis, thus decreasing turn-around time and increase a number of exams reviewed. By learning the physician's profile and physician preferences, the disclosed techniques may also identify similar profiles and apply certain preferences to other physicians with similar profiles. That is, the disclosed techniques may dynamically apply preferences of other similar users to improve an order of the list of clinical documents. By leveraging knowledge of similar physicians, the physician may learn about the information relied upon to make a diagnosis. In this way, training time may be reduced. Furthermore, the disclosed techniques may learn (e.g., via machine-learning routines) over time to customize and/or improve the order of the list of clinical documents. As such, the disclosed techniques may improve operations of determining the intent of the exam, identifying relevant clinical context documents, and dynamically creating a list of clinical context documents. Accordingly, the disclosed techniques may allow the physician reduce review time, reduce an amount of time needed to make a diagnosis and/or generate a report, increase review efficiency, and also increase quality of service delivered.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system comprising:
a processor-based device storing or accessing a ranking application, wherein the ranking application, when executed by the processor-based device, causes acts to be performed comprising:
determining a purpose of an exam request based on one or more attributes of the exam request;
retrieving a plurality of documents from a database based on the determined purpose;
assigning a relevancy score to each of the plurality of documents based on a user profile;
creating a list comprising the plurality of documents, wherein an order of the plurality of documents is based on the relevancy score; and
populating a graphical user interface (GUI) with the list for display on the processor-based device or another device.

2. The system of claim 1, wherein the one or more attributes of the exam request comprise patient demographics, a name of a referring physician, an institution of the referring physician, a date of an exam, a time of the exam, and a reason for the exam.

3. The system of claim 2, wherein the ranking application, when executed, causes acts to be performed comprising:
assigning the relevancy score to each of the plurality of documents based on the determined purpose of the exam request; and
adjusting the relevancy score of each of the plurality of documents based on the user profile.

4. The system of claim 1, wherein the ranking application is configured to identify one or more attributes of each document of the plurality of documents, wherein the one or more attributes comprise a type of the document, a name of a referring physician, an institution of the referring physician, a date of the document, a time of the document, a source of the document, a quality of the document, and a content of the document.

5. The system of claim 1, wherein the ranking application, when executed, causes acts to be performed comprising:
receiving user input of a search term from the GUI; and
adjusting the order of the plurality of documents of the list based on the search term.

6. The system of claim 1, wherein the user profile comprises workplace attributes, personal preference attributes, and role attributes.

7. The system of claim 6, wherein the workplace attributes comprise an institution name, an institution location, an institution size, and an institution type.

8. The system of claim 6, wherein the role attributes comprise a seniority level, a specialty, a modality, an experience level, and an activity type.

9. The system of claim 6, wherein the ranking application, when executed, causes acts to be performed comprising:
identifying one or more similar user profiles based on the workplace attributes of the user profile or the role attributes of the user profile; and
adjusting the relevancy score for each of the plurality of documents based on personal preferences of the one or more similar user profiles.

10. The system of claim 6, wherein the ranking application, when executed, causes acts to be performed comprising:
receiving user input adjusting the order of the plurality of documents; and
storing the adjustment as an additional personal preference attribute within the user profile.

11. The system of claim 1, wherein the order of the plurality of documents comprises a ranked ordering of the plurality of documents from a highest relevancy score to a lowest relevancy score.

12. A method comprising:
determining a purpose of an exam request based on attributes of the exam request;
assigning a relevancy score to each of a plurality of documents based on the determined purpose of the exam request;
adjusting the relevancy score for each of the plurality of documents based on a user profile;
creating a list of the plurality of documents, wherein the plurality of documents is ranked from a highest relevancy score to a lowest relevancy score; and
populating a graphical user interface (GUI) with the list of the plurality of documents for display on a workstation.

13. The method of claim 12, comprising:
adjusting a ranking of the plurality of documents based on a search term; and
storing the search term in the user profile.

14. The method of claim 12, comprising:
adjusting a ranking of the plurality of documents based on user input; and
storing the adjustment as a preference in the user profile.

15. The method of claim 12, comprising:
identifying one or more similar user profiles based on the user profile, wherein the user profile comprises workplace attributes, personal preference attributes, and role attribute; and
adjusting the relevancy score based on the one or more similar user profiles.

16. The method of claim 12, wherein assigning the relevancy score to each of the plurality of documents based on the purpose of the exam request comprises:
comparing one or more attributes of the exam request and one or more attributes of each document of the plurality of documents;
identifying a number of overlapping attributes between the exam request and each document of the plurality of documents; and
assigning the relevancy score based on the number of overlapping attributes.

17. A non-transitory, computer-readable medium comprising computer-readable code, that when executed by one or more processors, causes the one or more processors to perform operations comprising:
identifying a plurality of documents based on attributes of an exam;
assigning a relevancy score to each of the plurality of documents based on a user profile;
creating a list comprising the plurality of documents based on the relevancy score, wherein the plurality of documents is ordered from a highest relevancy score to a lowest relevancy score; and
populating a graphical user interface (GUI) with the list for display on a workstation.

18. The non-transitory, computer-readable medium of claim 17, wherein the operations comprise:
identifying one or more similar user profiles based on one or more attributes of the user profile; and
adjusting the relevancy score based on the one or more similar user profiles.

19. The non-transitory, computer-readable medium of claim 18, wherein the operations comprise adjusting the relevancy score based on a user input, wherein the user input comprises a search term or an indication to reorder a document of the plurality of documents.

20. The non-transitory, computer-readable medium of claim 19. wherein the operations comprise:
storing the search term or the indication in the user profile; and
populating a second GUI with the search term or the indication for display on the workstation.
